(19) United States
(12) Patent Application Publication
(10) Pub. No.: US 2010/0004569 A1
(43) Pub. Date: Jan. 7, 2010

(54) Pack for Eye Warming Massage

(76) Inventors: SuJung Kim, Seoul (KR); EunSook Yun, Seoul (KR); JooHo Kim, Gunpo-si (KR); ByungYoung Kang, Seoul (KR); YunYoung Ko, Seoul (KR); SoonJae Won, Hanam-si (KR); Grace Junghwang, Mountain View, CA (US); Daniel-Sung-hwe Kim, Mountain View, CA (US); SorenStephen Deorlow, San Francisco, CA (US); Lukas Martin Scherrer, San Francisco, CA (US)

Correspondence Address: PARK LAW FIRM, 3255 WILSHIRE BLVD, SUITE 1110, LOS ANGELES, CA, 90010, US

(21) Appl. No.: 12/375,667

(22) PCT Filed: Jul. 31, 2007
(86) PCT No.: PCT/KR2007/003665

§ 371 Date: Jan. 29, 2009

(30) Foreign Application Priority Data
Aug. 1, 2006 (KR) 20-2006-0020665

(51) Int. Cl. A61F 7/02

(52) U.S. Cl. 601/17; 607/109

(57) ABSTRACT

An eye warming pack warming eyes capable for warming to release fatigue of eyes and steaming a circumference of eyes is provided, The eye warming pack according to the present invention includes a heating portion where a heating material is packed in a pack, in which the heating material generates a heat energy when it returns to a solid state after it is converted to a liquid state by the operation of a piezoelectric element and a massage sheet containing a cosmetic massage lotion and being warmed by the heat energy from the heating portion.

### TECHNICAL FIELD

The present invention relates to an eye pack, more particularly, relates to an eye warming pack capable of warming eyes to release fatigue of eyes and steaming a circumference of eyes, where aging of skin can be progressed easily because secretion of sebum is less than other parts of a face, for example eyelid, under part of eyes, side part of eyes, etc, with a massage lotion which shows effects for protecting the skin to keep the skin elastic and healthy.

### BACKGROUND ART

Now a days, people lives on the benefits of the development of industrial society. However, it causes a lot of nuisance.

As one of nuisances, imbalance of the biorhythm can be exampled. It is well known that the imbalance of the biorhythm is caused by the influences of colorful lightings and electric devices.

Imbalance of biorhythm can cause xerophthalmia which is a drying effect of eye, and fatigue of eyes.

It is known that increase of time to use computers in the workplace and the house causes the xerophthalmia.

Although cumulative fatigue may be solved by medicines, there are troublesome to visit a hospital and to pay a time.

Further it is reasonable to cure a disease before the fatigue becomes accumulated.

Auxiliary medicines are used to release the fatigue of eyes. However, because auxiliary medicines must be applied to the eyes directly, it may be counterproductive when it is used consistently.

### DISCLOSURE OF INVENTION

### Technical Problem

An object of the present invention is to provide an eye warming pack capable of warming eyes to release fatigue of eyes.

Another object of the present invention is to provide an eye warming pack capable of massaging the circumference of the eyes with a cosmetic massaging lotion to keep the clearness of skin and slow the drooping of the skin and the development of fine wrinkles.

### Technical Solution

An eye warming pack according to the present invention includes:

a heating portion where a heating material is packed in a pack, in which the heating material generates a heat energy when it returns to a solid state after it is converted to a liquid state by the operation of a piezoelectric element; and

a massage sheet containing a cosmetic massage lotion and being warmed by the heat energy from the heating portion.

The piezoelectric element converts mechanical energy to electric energy so that the heating material is converted into the liquid state and the cosmetic massage lotion, which contained in the massaging sheet, is evaporated while the massaging sheet is warmed by the heat energy from the heating portion so that the evaporated cosmetic massage lotion steams the circumference of eyes.

### Advantageous Effects

The eye warming pack according to the present invention can release the fatigue of the eyes by warming the eyes and performs an effective skin care around the eyes.

According to the eye warming pack according to the present invention. Clearness of the skin can be maintained and drooping of the skin and development of fine wrinkle can be slowed, and healthy skin can be maintained.

### BRIEF DESCRIPTION OF THE DRAWINGS

The above and other features and advantages of the present invention will become more apparent by describing in detail preferred embodiments thereof with reference to the attached drawings in which:

FIG. 1 is a perspective view of the eye warming pack according to the present invention being packed by a packing band;

FIG. 2 is a front view of the eye warming pack according to the present invention when it is packed in the packing band;

FIG. 3 is an exemplary view showing the state when a heat energy is generated from the heating material according to the present invention;

FIG. 4 is an exemplary view showing the state when the eye warming pack is pulled out from the packing band; and

FIG. 5 is an exemplary view showing the state when eyes are warmed by contacting the eye warming pack to the circumference of the eyes.

### BRIEF DESCRIPTION OF NUMERALS IN DRAWINGS

10 . . . heating portion

11 . . . vinyl pack

12 . . . heating material

13 . . . piezoelectric element

20 . . . massage sheet

30 . . . packing band

### MODE FOR THE INVENTION

Reference will now be made in detail to the embodiments of the present invention, examples of which are illustrated in the accompanying drawings, wherein like reference numerals refer to the like elements throughout. The embodiments are described below to explain the present invention by referring to the figures. For the sake of clearness and concision, technology related to the present invention that is not novel and is well known in the art to which present invention pertains will not be described herein.

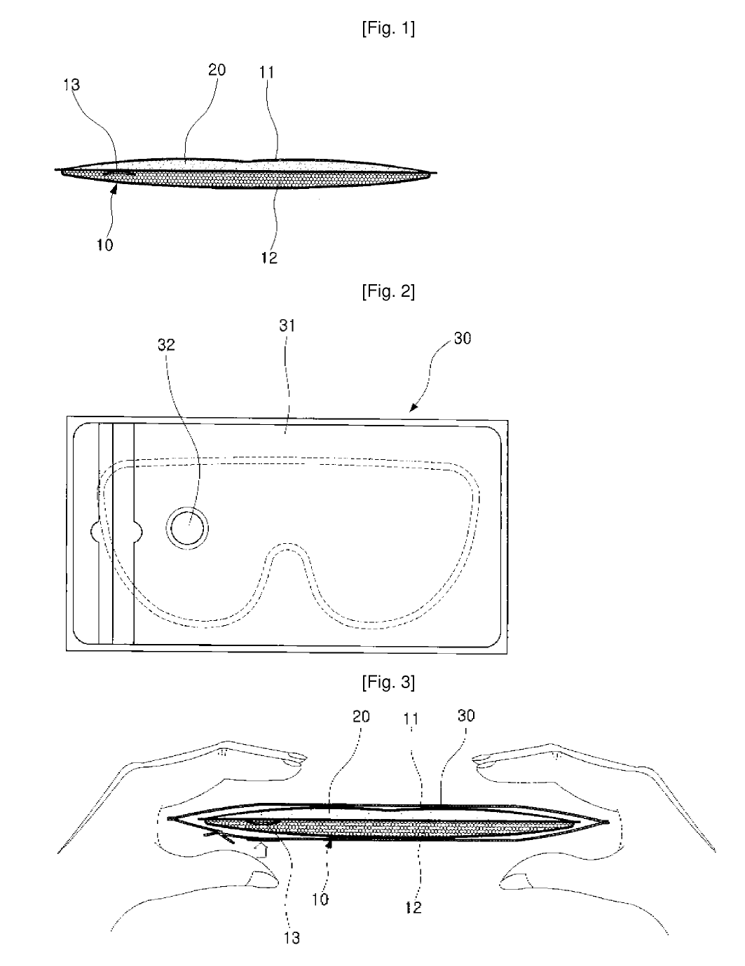

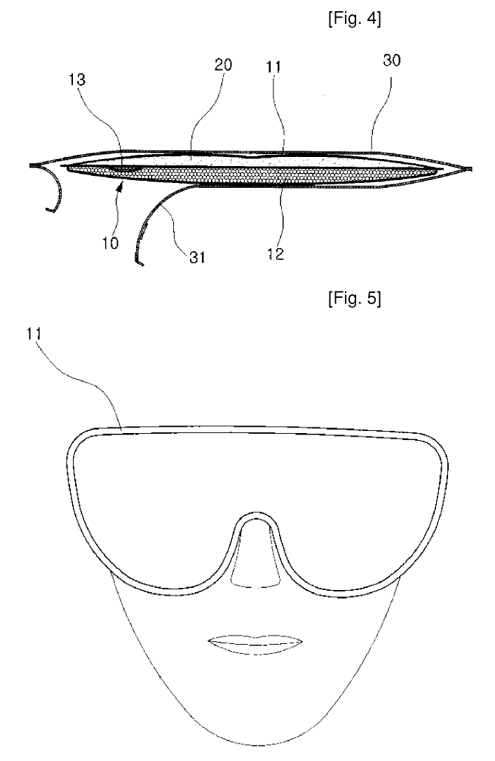

As shown in FIG. 1 through FIG. 5, the eye warming pack according to the present invention includes a heating portion 10, a massage sheet 20 which is warmed by a heat energy from the heating portion 10 and a packing band 30.

The heating portion 10 is comprised of a vinyl pack 11 and a heating material 12. The heating material 12 generates a heat energy when it returns from a liquid state to a solid state after it is converted to the liquid state.

Also a piezoelectric element 13 is contained in the vinyl pack 11. The piezoelectric element 13 converts a mechanical energy to an electric energy so that the heating material 12 can be converted from a solid state to a liquid state.

The heating material can be one of a sodium thiosulfate (Na2S2O3) and a sodium acetate. The sodium acetate is called commonly as “HYPO”.

The heating material exists as a solid state in a room temperature. However, the heating material generates heat energy when it returns from a liquid state to a solid state after it is converted into a liquid state.

The piezoelectric element 13, which is packed together with the heating material 12 in the vinyl pack 11, is conductive. The piezoelectric element 13 has a elasticity so that mechanical movement can be available. The piezoelectric element 13 is one of solid materials which can convert the given mechanical energy to an electric energy.

The massage sheet 20 is warmed by the heat energy from the heating portion 10. The vinyl pack 11 and the massage sheet 20 are provided as an unit by sewing edges of the massage sheet 20 to the vinyl pack 20.

The massage sheet 20 can be made one of a pulp and a fabric.

A cosmetic massage lotion is contained in the massage sheet 20. The cosmetic massage lotion is evaporated while the massage sheet 20 is warmed by the heat energy from the heating portion 10 so that the evaporated cosmetic massage lotion can steam the circumference of the eyes.

A pressing mark 32 is noted in the front side of the eye warming pack so that the location of the piezoelectric element 13 can be identified easily. The eye warming pack is packed to a packing band 30 which has a front panel 20. The front panel 20 can be unfolded in both ways.

Hereinafter, the usage of the eye warming pack according to the present invention is described in detail.

While the eye warming pack according to the present invention is packed in the pac king band 30, the massage sheet 20 is warmed by the heat energy from the heating portion 10.

The piezoelectric element 13 moves mechanically when it is turned over repeatedly by hands. When turning over, a thumb is located at the pressing mark 32, which is noted on the front panel 31, and the others are located at the rear of the front panel 31, as shown in FIG. 3.

Then, the heating material 12 of a solid state is converted into a liquid state when a mechanical energy of the piezoelectric element 13 is converted into an electric energy. Usually, the heating material 12 maintains a solid state in a room temperature.

A lot of heat energy is generated when the heating material 12, which is converted to a liquid state by the operation of the piezoelectric element 13, is reversed to a solid state. Then, the massage sheet 20 becomes warmed by the heat energy from the heating portion 10.

After the massage sheet 20 is warmed by operating the piezoelectric element 13, the eye warming pack is pulled out by opening the front panel 31 of the packing band 30.

warming and steaming of eyes can be done by contacting the eye warming pack which is pulled out from the packing band 30, At this time, the massage sheet 20 is contacted to the circumference of eyes so that the massage sheet 20 can be contacted to the skin around the eyes.

The cosmetic massage lotion, which is contained in the massage sheet 20, is evaporated from the massage sheet 20 by the heat energy from the heating portion 10. The evaporated cosmetic massage lotion warms eyes so that the fatigue of eyes can be released. Further, the cosmetic massage lotion soaks through the skin so that skin care can be performed.

### INDUSTRIAL APPLICABILITY

An eye pack warms eyes to release fatigue of eyes and steams a circumference of eyes with a massage lotion which shows effects for protecting the skin to keep the skin elastic and healthy.

1. An eye warming pack comprising;
a heating portion where a heating material is packed in a pack, in which the heating material generates a heat energy when it returns to a solid state after it is converted to a liquid state by the operation of a piezoelectric element; and
a massage sheet containing a cosmetic massage lotion and being warmed by the heat energy from the heating portion,
in which the piezoelectric element converts mechanical energy to electric energy so that the heating material is converted into the liquid state and the cosmetic massage lotion, which contained in the massaging sheet, is evaporated while the massaging sheet is warmed by the heat energy from the heating portion so that the evaporated cosmetic massage lotion steams the circumference of eyes.

2. The eye warming pack in claim 1, in which the heating material is a sodium thiosulfate (Na2S2O3) or a sodium acetate.

3. The eye warming pack in claim 1, further comprising a packing band for packing,
in which a pressing mark to identify the location of the piezoelectric element is marked in the front side of the packing band.
